(19) United States
(12) Patent Application Publication
(10) Pub. No.: US 2023/0085243 A1
(43) Pub. Date: Mar. 16, 2023

(54) METHOD FOR IMPROVING THE HUE OF RECYCLED BIS(2-HYDROXYETHYL) TEREPHTHALATE

(71) Applicant: NAN YA PLASTICS CORPORATION, Taipei (TW)

(72) Inventors: TE-CHAO LIAO, TAIPEI (TW); JUNG-JEN CHUANG, TAIPEI (TW); ZHANG-JIAN HUANG, TAIPEI (TW); Yu-Ti Tseng, TAIPEI (TW); CHENG-HSUN CHEN, TAIPEI (TW)

(21) Appl. No.: 17/864,354

(22) Filed: Jul. 13, 2022

(30) Foreign Application Priority Data
Sep. 13, 2021 (TW) 110133988

(51) Int. Cl. C08J 11/24; B01J 20/20; B01J 20/28; C07C 67/54

(52) U.S. Cl. CPC C08J 11/24; B01J 20/20; B01J 20/28061; B01J 20/28064; B01J 20/28066; B01J 20/28071; B01J 20/28073; B01J 20/28076; C07C 67/54; C07B 2200/13; C08J 2367/02

(57) ABSTRACT

A method for improving the hue of recycled BHET is provided. The method includes: providing a recycled polyester fabric; using a chemical de-polymerization liquid to chemically depolymerize the recycled polyester fabric to form a de-polymerization product; distilling out the chemical de-polymerization liquid from the de-polymerization product by evaporation; dissolving the BHET in water to form a aqueous phase liquid; adding an activated carbon material to the aqueous phase liquid to adsorb impurities; and cooling the aqueous phase liquid to crystallize the BHET from the aqueous phase liquid to obtain a recycled BHET.

### CROSS-REFERENCE TO RELATED PATENT APPLICATION

This application claims the benefit of priority to Taiwan Patent Application No. 110133988, filed on Sep. 13, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a method for recycling a polyester material, and more particularly to a method for improving the hue of recycled bis(2-hydroxyethyl) terephthalate (BHET).

### BACKGROUND OF THE DISCLOSURE

In the related art, a chemical recycling method of a PET fabric primarily uses a chemical de-polymerization liquid (e.g., ethylene glycol) to chemically depolymerize the PET fabric, so as to form a de-polymerization product. The de-polymerization product mainly contains bis(-2-hydroxyethyl) terephthalate (BHET). However, the aforementioned chemical recycling method requires complicated purification procedures to remove impurities (such as dyes) originally present in the PET fabric, so that the BHET can be repolymerized to form high-quality recycled polyester chips (r-PET).

In the aforementioned BHET purification procedures, a conventional purification technology is to use activated carbon or an ion exchange resin to adsorb the impurities (such as dyes) in a crude BHET product that contains ethylene glycol (EG), or separates out BHET by distillation.

However, the aforementioned two manners of purification both have the disadvantages such as a poor recovery quality (e.g., poor hue) and a high recovery cost of BHET.

A method for depolymerizing PET fabric is provided in the U.S. Pat. Publication Ser. No. 9,255,194. Although the method provided in the said patent can completely remove dyes and other impurities while recycling catalysts, the method still requires complicated purification procedures for BHET purification, thereby causing a low recovery rate and a poor recovery quality of BHET.

A method for depolymerizing PET fabric is provided in the China Patent Application No. 100,344,604, and the method also requires complicated purification procedures for BHET purification, thereby causing an exceedingly high recovery cost of materials and a poor recovery quality of BHET.

Therefore, how to improve on the aforementioned technical inadequacies has become an issue to be addressed in the relevant field.

### SUMMARY OF THE DISCLOSURE

A technical problem to be solved by the present disclosure is to provide a method for improving the hue of BHET in response to the disadvantages of the prior art.

In one aspect, the present disclosure provides a method for improving the hue of recycled bis(2-hydroxyethyl) terephthalate (BHET). The method includes: providing a recycled polyester fabric attached with impurities; performing a de-polymerization process that includes using a chemical de-polymerization liquid to chemically depolymerize the recycled polyester fabric to form a de-polymerization product containing BHET, the chemical de-polymerization liquid, and the impurities; performing an evaporation process that includes: distilling out the chemical de-polymerization liquid from the de-polymerization product by evaporation so that the BHET is separated from the chemical de-polymerization liquid; performing an adsorption process that includes: dissolving the BHET in water to form a aqueous phase liquid and adding an activated carbon material to the aqueous phase liquid so that the activated carbon material adsorbs the impurities originally present in the recycled polyester fabric to increase the purity of the BHET in the aqueous phase liquid; and performing a crystallization process that includes: cooling the aqueous phase liquid to crystallize the BHET from the aqueous phase liquid to obtain a recycled BHET.

In certain embodiments, the recycled BHET has an L value of not less than 90, an a value of between -2 and 2, a b value of between -4 and 4, and a recovery rate of not less than 85%.

In certain embodiments, in the de-polymerization process, the chemical de-polymerization liquid is used to chemically depolymerize the recycled polyester fabric in the presence of a de-polymerization catalyst, in which the de-polymerization catalyst is a metal catalyst, and the chemical de-polymerization liquid is ethylene glycol (EG).

In certain embodiments, in the de-polymerization process, the chemical de-polymerization liquid is heated to a de-polymerization temperature to chemically depolymerize the recycled polyester fabric, in which the de-polymerization temperature is between 180° C. and 260° C.

In certain embodiments, in the evaporation process, the de-polymerization product is heated to an evaporation temperature to distill out the chemical de-polymerization liquid from the de-polymerization product, in which the chemical de-polymerization liquid is ethylene glycol (EG), and the evaporation temperature is between 150° C. and 250° C.

In certain embodiments, in the adsorption process, a specific surface area of the activated carbon material is between 400 m²/g. and 4,000 m²/g.

In certain embodiments, in the adsorption process, a pH value of the activated carbon material is between 4 and 7.

In certain embodiments, in the adsorption process, a micropore volume of the activated carbon material is between 0.20 ml/g and 2.00 ml/g.

In certain embodiments, in the adsorption process, a weight ratio of the BHET to the water ranges from 1:3 to 1:20.

In certain embodiments, in the adsorption process, a weight ratio of the activated carbon material to the BHET ranges from 1:10 to 1:200.

In certain embodiments, in the adsorption process, the aqueous phase liquid is heated to an adsorption temperature so that the activated carbon material adsorbs the impurities at the adsorption temperature, in which the adsorption temperature is between 70° C. and 150° C.

In certain embodiments, in the crystallization process, the aqueous phase liquid is cooled from the adsorption temperature to a crystallization temperature to crystallize the BHET from the aqueous phase liquid, in which the crystallization temperature is between 5° C. and 25° C., that is, a terminal temperature is between 5° C. and 25° C.

In certain embodiments, in the adsorption process, the adsorption temperature is between 80° C. and 130° C.

Therefore, in the method for improving the hue of recycled BHET provided by the present disclosure, the recovery quality and recovery rate of the recycled BHET can be improved through the technical solution of “performing a de-polymerization process that includes: using a chemical de-polymerization liquid to chemically depolymerize the recycled polyester fabric to form a de-polymerization product containing BHET, the chemical de-polymerization liquid, and the impurities; performing an evaporation process that includes: distilling out the chemical de-polymerization liquid from the de-polymerization product by evaporation so that the BHET is separated from the chemical de-polymerization liquid; performing an adsorption process that includes: mixing water with the BHET and the impurities to form an aqueous phase liquid, and adding an activated carbon material to the aqueous phase liquid so that the activated carbon material adsorbs the impurities to improve the purity of the BHET in the aqueous phase liquid; and performing a crystallization process, including: cooling the aqueous phase liquid to crystallize the BHET from the aqueous phase liquid to obtain a recycled BHET”. Further, the method provided in the present disclosure has an advantage of having a low cost.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a method for improving the hue of recycled BHET according to an embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### Method for Improving the Hue of Recycled BHET

Common polyester fabric is often attached with impurities such as dyes and water repellents. In order to recycle polyester fabric, conventionally, a chemical de-polymerization liquid (e.g., ethylene glycol) is primarily used to chemically depolymerize the polyester fabric to form a de-polymerization product that mainly includes bis(-2-hydroxyethyl) terephthalate (BHET).

Further, in order to purify BHET, a conventional purification manner uses activated carbon or ion exchange resin to adsorb impurities such as dyes in a crude BHET product containing ethylene glycol (EG), and then adds water to crystallize BHET. However, the BHET obtained by such a purification manner has poor hue and quality (with a maximum L value of only substantially 80, an a value of substantially -4 to 4, and a b value of substantially -6 to 6, and a maximum yield of BHET is only substantially 80%.

Another conventional purification technology separates out BHET by three distillations. This purification manner results in an exceedingly high equipment cost (as additional three film evaporators are needed), and a fairly low yield of BHET (only substantially 65%).

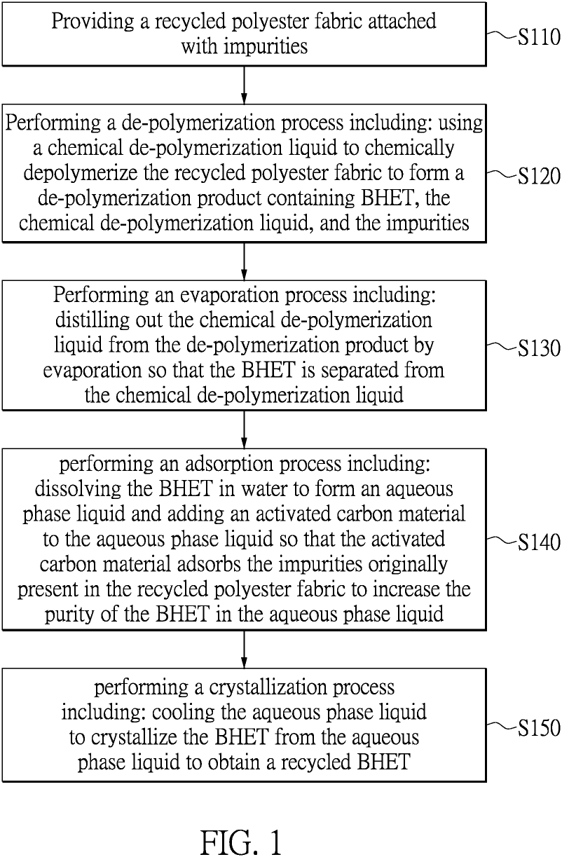

In order to solve the aforementioned technical problems, referring to FIG. 1, an embodiment of the present disclosure provides a method for improving the hue of recycled BHET. The method can effectively improve the recovery quality and yield of the recycled BHET, and has an advantage of a low cost. Further, the method includes step S110, step S120, step S130, step S140 and step S150. Notably, sequences of the steps and practical process modes of this embodiment can be adjusted according to requirements and are not limited thereto.

The step S110 includes: providing a recycled polyester fabric attached with impurities, in which the impurities can be, for example, dyes or water repellents, but the present disclosure is not limited thereto.

For instance, the recycled polyester fabric can obtain color (e.g., black, red and blue) by being dyed with a dye. Further, the recycled polyester fabric can obtain water-repellent function through treatment using a water repellent.

The dye can be, for example, at least one of natural dyes and synthetic dyes, or at least one of physical dyes and chemical dyes.

Further, the water repellent can have, for example, a polymer crosslinked structure and can be, for example, a water repellent containing silicon (Si), a water repellent containing fluorine (F), a water repellent containing fluorine and silicon, or a waterborne polyurethane (PU) water repellent, but the present disclosure is not limited thereto.

In one embodiment of the present disclosure, the recycled polyester fabric is dyed to obtain an L value of greater than 0 and not greater than 30, that is, the recycled polyester fabric has a relatively darker color, but the present disclosure is not limited thereto. It should be noted that, the aforementioned L value is a parameter value for expressing brightness (also known as whiteness of color) in a Lab color space.

The step S120 includes: performing a de-polymerization process. The de-polymerization process includes: using a chemical de-polymerization liquid to chemically depolymerize the recycled polyester fabric to form a de-polymerization product containing bis(-2-hydroxyethyl) terephthalate (BHET), oligomers, the chemical de-polymerization liquid, and the impurities.

More specifically, the chemical de-polymerization liquid can be, for example, ethylene glycol (EG); and a method for chemically depolymerizing the recycled polyester fabric can be, for example, an EG de-polymerization method so that the recycled polyester fabric can be depolymerized to form a de-polymerization product mainly containing BHET. Further, the de-polymerization product also contains oligomers formed by depolymerizing the polyester fabric, the aforementioned chemical de-polymerization liquid for de-polymerization, and impurities originally present in the recycled polyester fabric.

Notably, BHET is an intermediate between purified terephthalic acid (PTA) and ethylene glycol (EG). Further, BHET also can be used as a raw material for synthesizing polyester (PET), and still can be generated with other monomers into polyester copolymers.

In an embodiment of the present disclosure, the chemical de-polymerization liquid is used to chemically depolymerize the recycled polyester fabric in the presence of a de-polymerization catalyst, in which the de-polymerization catalyst can be, for example, a metal catalyst, but the present disclosure is not limited thereto. Notably, the de-polymerization catalyst can assist in reducing the activation energy of the chemical de-polymerization liquid chemically depolymerizing the polyester fabric. From another perspective, the de-polymerization catalyst can assist in improving the efficiency of the chemical de-polymerization liquid chemically depolymerizing the recycled polyester fabric.

In one embodiment of the present disclosure, the metal catalyst can be, for example, at least one material selected from a group consisting of zinc acetate, lead acetate, cadmium acetate, calcium acetate, barium acetate, sodium acetate, lithium hydroxide, mercury acetate, copper acetate, and iron acetate, but the present disclosure is not limited thereto.

Or, in one embodiment of the present disclosure, the metal catalyst can be, for example, an organo titanium metal catalyst; or, in one embodiment of the present disclosure, the metal catalyst can be, for example, an ionic liquid catalyst, but the present disclosure is not limited thereto.

In one embodiment of the present disclosure, the chemical de-polymerization liquid is heated to a de-polymerization temperature to chemically depolymerize the recycled polyester fabric, where the de-polymerization temperature is preferably between 180° C. and 260° C., and more preferably between 190° C. and 240° C. At the aforementioned de-polymerization temperature, the efficiency of the chemical de-polymerization liquid chemically depolymerizing the recycled polyester fabric can be improved in a more effective manner, and the metal catalyst can exert a greater catalytic effect.

In one embodiment of the present disclosure, a de-polymerization pressure of the chemical de-polymerization liquid chemically depolymerizing the recycled polyester fabric is between 1.0 bar and 3.0 bar. In addition, a de-polymerization time of the chemical de-polymerization liquid chemically depolymerizing the recycled polyester fabric is between 1.0 hour and 8.0 hours.

The step S130 includes: performing an evaporation process. The evaporation process includes: distilling out the chemical de-polymerization liquid from the de-polymerization product by evaporation so that the BHET is separated from the chemical de-polymerization liquid.

More specifically, in the evaporation process, the de-polymerization product is heated to an evaporation temperature to distill out the chemical de-polymerization liquid from the de-polymerization product, in which the chemical de-polymerization liquid is ethylene glycol (EG). Further, the evaporation temperature is preferably between 150° C. and 250° C., and more preferably between 160° C. and 220° C.

Notably, in the de-polymerization product formed by the de-polymerization process (step S120), a boiling point of the chemical de-polymerization liquid is often lower than a boiling point of the BHET. Specifically, the boiling point of the chemical de-polymerization liquid is approximately between 180° C. and 220° C., and the boiling point of the BHET is approximately between 380° C. and 420° C., but the present disclosure is not limited thereto.

Accordingly, the evaporation process can distill out the chemical de-polymerization liquid having a lower boiling point from the de-polymerization product by evaporation based on a difference in the boiling points of different components in the mixed liquid. Therefore, the purity of BHET in the de-polymerization product can be effectively improved. Notably, the impurities originally present in the recycled polyester fabric remains in the de-polymerization product after the evaporation process, and these impurities need a follow-up adsorption process to be removed.

The step S140 includes: performing an adsorption process. The adsorption process includes: dissolving the BHET in water to form an aqueous phase liquid. Then, the adsorption process further includes: adding an activated carbon material to the aqueous phase liquid so that the activated carbon material can be used to adsorb the impurities (e.g., organic dyes and coloring substances) originally present in the recycled polyester fabric. Accordingly, the impurities can be removed from the de-polymerization product, and the purity of the BHET in the aqueous phase liquid can be effectively improved.

Notably, the activated carbon material is a porous carbon-containing substance and has a highly developed pore structure. In addition to carbon, the composition of the activated carbon material also contains a small amount of hydrogen, nitrogen, oxygen, and ash. The structure of the activated carbon material is formed by stacking six-membered rings of carbon. An irregular arrangement of the six-membered rings of carbon results in the features of a large micropore volume and a large surface area of the activated carbon material. The activated carbon material is insoluble in water or organic solvents. The activated carbon material has a strong adsorption capacity for organic polymer substances (e.g., organic dyes and coloring substances). An adsorption effect of the activated carbon material is realized by a physical adsorption capacity and a chemical adsorption capacity.

In one embodiment of the present disclosure, in order to improve the adsorption efficiency of the activated carbon material for the impurities (e.g., organic dyes), a specific surface area of the activated carbon material is preferably between 400 m²/g and 4,000 m²/g, and more preferably between 800 m²/g and 2,000 m²/g. A pH value of the activated carbon material is preferably between 4 and 7, and more preferably between 5 and 6.5. Further, a micropore volume of the activated carbon material is preferably between 0.20 ml/g and 2.00 ml/g, and more preferably between 0.80 ml/g and 1.50 ml/g.

In one embodiment of the present disclosure, in order to improve the adsorption efficiency of the activated carbon material for the impurities (e.g., organic dyes), a weight ratio of the BHET to the water is preferably 1:3 to 1:20, and more preferably 1:4 to 1:15.

That is to say, in the aqueous phase liquid, the weight of the water is preferably 3 to 20 times the weight of the BHET, and more preferably 4 to 15 times, but the present disclosure is not limited thereto.

In one embodiment of the present disclosure, in order to improve the adsorption efficiency of the activated carbon material for the impurities (e.g., organic dyes), a weight ratio of the activated carbon material to the BHET is preferably 1:10 to 1:200, and more preferably 1:20 to 1:150.

That is to say, in the aqueous phase liquid, the weight of the BHET is preferably 10 to 200 times the weight of the activated carbon material, and more preferably 20 to 150 times, but the present disclosure is not limited thereto.

In one embodiment of the present disclosure, in order to improve the adsorption efficiency of the activated carbon material for the impurities (e.g., organic dyes), the aqueous phase liquid is heated to an adsorption temperature so that the activated carbon material adsorbs the impurities at the adsorption temperature, in which the adsorption temperature is preferably between 70° C. and 150° C., and more preferably between 80° C. and 130° C. Notably, it has been observed that when the adsorption temperature is between 80° C. and 130° C., the activated carbon material has high adsorption efficiency for the impurities.

In one embodiment of the present disclosure, the adsorption process (step S140) is further defined to be performed after the evaporation process (step S130). That is to say, the chemical de-polymerization liquid in the de-polymerization product is firstly distilled out by evaporation so that the BHET is firstly separated from the chemical de-polymerization liquid (e.g., EG). Then, the impurities (e.g., organic dyes) are further adsorbed by the activated carbon material in the aqueous phase liquid for removal. Therefore, the recycled BHET obtained by the subsequent steps has good quality and hue.

In addition, notably, in order to facilitate a subsequent crystallization process, the activated carbon material can be firstly filtered with a filter screen so that the BHET is separated from the activated carbon material attached with impurities.

The step S150 includes: performing a crystallization process. The crystallization process includes: cooling the aqueous phase liquid to crystallize the BHET from the aqueous phase liquid to obtain a recycled BHET.

In one embodiment of the present disclosure, the aqueous phase liquid is cooled from the adsorption temperature (e.g., 70° C. to 150° C.) to a crystallization temperature to crystallize the BHET from the aqueous phase liquid to obtain a solid recycled BHET, in which the crystallization temperature is preferably between 5° C. and 25° C., that is, a terminal temperature is between 5° C. and 25° C.

For instance, the aqueous phase liquid can be cooled, for example, from an adsorption temperature of 150° C. to a crystallization temperature of 25° C., or the aqueous phase liquid can be cooled, for example, from an adsorption temperature of 100° C. to a crystallization temperature of 5° C., to crystallize the BHET from the aqueous phase liquid.

According to the aforementioned configuration, the recycled BHET has a good hue, a good recovery quality, and a high recovery rate. Further, the method provided by the embodiments of the present disclosure has an advantage of a low cost. Specifically, the recycled BHET has an L value of not less than 90, an a value of between -2 and 2, and a b value of between -4 and 4, as well as a recovery rate of not less than 85%.

Notably, a Lab color space is a color component space, with a dimension L expressing brightness, and a and b expressing color component dimensions, as well as CIE XYZ color space coordinates based on nonlinear compression.

### Experimental Data and Test Results

In order to prove that the method for improving the hue of recycled BHET provided by embodiments of the present disclosure has a good recovery effect and a good hue improvement effect, Examples 1 to 3 and Comparative examples 1 to 3 are exemplified below for description.

### Example 1

1 kg of a white PET fabric, 6 kg of ethylene glycol and 20 g of a zinc acetate catalyst were put into a 10 L three-neck glass bottle, heated to 190° C. and stirred for 6 hours; then, the mixture was heated until the reaction was maintained boiling (195° C. to 210° C.); and surplus EG was distilled out so that the amount of residual EG in the reaction was less than 5%.

After the reaction was cooled to 90° C., 18 kg of water was added to the mixture, and the mixture was heated to 90° C. so that BHET was dissolved in the water; 30 g of activated carbon was added, and the resulting mixture was stirred for 1 hour while the temperature was maintained at 90° C. to adsorb impurities such as dyes; then, the activated carbon and the impurities were filtered out, and the resulting transparent aqueous solution was cooled to 5° C. to precipitate BHET; and then, the BHET was filtered and dried.

Quality of BHET: L = 92%, a = 1.4, b = 2.4, with a yield of 90.0%.

### Example 2

Compared with Example 1, the temperature for the activated carbon to adsorb the impurities such as dyes was 95° C. instead of 90° C.

Quality of BHET: L = 91%, a = 1.5, b = 2.6, with a yield of 89.4%.

### Example 3

Compared with Example 1, the temperature for the activated carbon to adsorb the impurities such as dyes was 85° C. instead of 90° C. Quality of BHET: L = 91%, a = 0.7, b = 2.7, with a yield of 89.7%.

### Example 4

Compared with Example 1, the temperature for the activated carbon to adsorb the impurities such as dyes was 125° C. instead of 90° C. Quality of BHET: L = 92%, a = 0.1, b = 1.3, with a yield of 86.2%.

### Comparative Example 1

1 kg of a white PET fabric, 6 kg of ethylene glycol and 20 g of a zinc acetate catalyst were put into a 10 L three-neck glass bottle, heated to 190° C. and stirred for 6 hours; then, the mixture was heated until the reaction was maintained boiling (195° C. to 210° C.); and surplus EG was distilled out so that the amount of residual EG in the reaction was less than 5%.

After the reaction was cooled to 90° C., 18 kg of water was added, and the mixture was heated to 65° C. so that BHET was dissolved in the water; 30 g of activated carbon was added, and the resulting mixture was stirred for 1 hour while the temperature was maintained at 75° C. to adsorb impurities such as dyes; then, the activated carbon and the impurities were filtered out, and the resulting transparent aqueous solution was cooled to 5° C. to precipitate BHET; and then, the BHET was filtered and dried.

Quality of BHET: L = 78%, a = 2.4, b = 7.0, with a yield of 89.0%.

### Comparative Example 2

Compared with Comparative example 1, the temperature for the activated carbon to adsorb the impurities such as dyes was 55° C. instead of 65° C.

Quality of BHET: L = 76%, a = 2.2, b = 7.4, with a yield of 60.0%.

### Comparative Example 3

Compared with Comparative example 1, the temperature for the activated carbon to adsorb the impurities such as dyes was 180° C. instead of 65° C.

Quality of BHET: L = 92%, a = 1.8, b = 3.4, with a yield of 78.0%.

### Beneficial Effects of the Embodiments

In conclusion, in the method for improving the hue of recycled BHET provided by the present disclosure, the recovery quality and recovery rate of the recycled BHET can be improved through the technical solution of “performing a de-polymerization process that includes: using a chemical de-polymerization liquid to chemically depolymerize the recycled polyester fabric to form a de-polymerization product containing BHET, the chemical de-polymerization liquid, and the impurities; performing an evaporation process that includes: distilling out the chemical de-polymerization liquid from the de-polymerization product by evaporation so that the BHET is separated from the chemical de-polymerization liquid; performing an adsorption process that includes: mixing water with the BHET and the impurities to form an aqueous phase liquid, and adding an activated carbon material to the aqueous phase liquid so that the activated carbon material adsorbs the impurities to improve the purity of the BHET in the aqueous phase liquid; and performing a crystallization process, including: cooling the aqueous phase liquid to crystallize the BHET from the aqueous phase liquid to obtain a recycled BHET”. Further, the method provided in the present disclosure has an advantage of having a low cost.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for improving the hue of recycled bis(2-hydroxyethyl) terephthalate (BHET), comprising:
providing a recycled polyester fabric attached with impurities;
performing a de-polymerization process including: using a chemical de-polymerization liquid to chemically depolymerize the recycled polyester fabric to form a de-polymerization product containing BHET, the chemical de-polymerization liquid, and the impurities;
performing an evaporation process including: distilling out the chemical de-polymerization liquid from the de-polymerization product by evaporation so that the BHET is separated from the chemical de-polymerization liquid;
performing an adsorption process including: dissolving the BHET in water to form an aqueous phase liquid and adding an activated carbon material to the aqueous phase liquid so that the activated carbon material adsorbs the impurities originally present in the recycled polyester fabric to increase the purity of the BHET in the aqueous phase liquid; and
performing a crystallization process including: cooling the aqueous phase liquid to crystallize the BHET from the aqueous phase liquid to obtain a recycled BHET.

2. The method according to claim 1, wherein the recycled BHET has an L value of not less than 90, an α value of between -2 and 2, a b value of between -4 and 4, and a recovery rate of not less than 85%.

3. The method according to claim 1, wherein in the de-polymerization process, the chemical de-polymerization liquid is used to chemically depolymerize the recycled polyester fabric in the presence of a de-polymerization catalyst, and wherein the de-polymerization catalyst is a metal catalyst, and the chemical de-polymerization liquid is ethylene glycol (EG).

4. The method according to claim 3, wherein in the de-polymerization process, the chemical de-polymerization liquid is heated to a de-polymerization temperature to chemically depolymerize the recycled polyester fabric, and wherein the de-polymerization temperature is between 180° C. and 260° C.

5. The method according to claim 1, wherein in the evaporation process, the de-polymerization product is heated to an evaporation temperature to distill out the chemical de-polymerization liquid from the de-polymerization product, and wherein the chemical de-polymerization liquid is ethylene glycol (EG), and the evaporation temperature is between 150° C. and 250° C.

6. The method according to claim 1, wherein in the adsorption process, a specific surface area of the activated carbon material is between 400 m²/g and 4,000 m²/g.

7. The method according to claim 1, wherein in the adsorption process, a pH value of the activated carbon material is between 4 and 7.

8. The method according to claim 1, wherein in the adsorption process, a micropore volume of the activated carbon material is between 0.20 ml/g and 2.00 ml/g.

9. The method according to claim 1, wherein in the adsorption process, a weight ratio of the BHET to the water ranges from 1:3 to 1:20.

10. The method according to claim 9, wherein in the adsorption process, a weight ratio of the activated carbon material to the BHET ranges from 1:10 to 1:200.

11. The method according to claim 1, wherein in the adsorption process, the aqueous phase liquid is heated to an adsorption temperature so that the activated carbon material adsorbs the impurities at the adsorption temperature, and wherein the adsorption temperature is between 70° C. and 150° C.

12. The method according to claim 11, wherein in the crystallization process, the aqueous phase liquid is cooled from the adsorption temperature to a crystallization temperature to crystallize out the BHET from the aqueous phase liquid, and wherein the crystallization temperature is between 5° C. and 25° C.
